(19) United States
(12) Patent Application Publication
(10) Pub. No.: US 2020/0129047 A1
(43) Pub. Date: Apr. 30, 2020

(54) A RIGID ENDOSCOPE

(71) Applicant: Arly Tingvatn, Bønes (NO)

(72) Inventor: Arly Tingvatn, Bønes (NO)

(21) Appl. No.: 16/626,803

(22) PCT Filed: Jun. 28, 2018
(86) PCT No.: PCT/NO2018/050170

§ 371 Date: Dec. 26, 2019

(30) Foreign Application Priority Data
Jun. 30, 2017 (NO) 20171085
Nov. 1, 2017 (NO) 20171736

(51) Int. Cl. A61B 1/00; A61B 1/303

(52) U.S. Cl. CPC A61B 1/00103; A61B 1/303; A61B 1/00034; A61B 1/00096; A61B 1/00013

(57) ABSTRACT

A rigid endoscope, preferably a disposable endoscope, with a rigid tubular body comprises a separate observation channel having a closed end, into which an optical instrument (e.g. a camera) may be introduced. The rigid endoscope may be an anoscope, a rectoscope, a vaginoscope or an amnioscope.

### FIELD OF THE INVENTION

The invention concerns the field of rigid endoscopes. More particularly, the invention concerns a rigid endoscope as set out by the preamble of claim 1 and a rigid endoscope system as set out by the preamble of claim 9.

### BACKGROUND OF THE INVENTION

Endoscopes for direct visual examination of the digestive tract are well known. Flexible endoscopes, such as colonoscopes and flexible sigmoidoscopes, are normally used for examination of the colon between the cecum and the descending colon, while rigid endoscopes are used for examining the lower sigmoid colon, rectum and anus. The invention is concerned with rigid endoscopes, of which four types are commonly known: anoscopes (also referred to as proctoscopes), rectoscopes, vaginoiscopes and amnioscopes. Anoscopes are designed for examination of the anus, while rectoscopes (sometimes referred to as rigid sigmoidoscopes) are designed for examination of the rectum and lower parts of the sigmoid colon. The vaginoscope is designed for examinations of the vagina and amnioscope is introduced into the cervical canal, permits direct visualizations of the fetus and amniotic fluid. A typical anoscope may have a length of approximately 10 cm, while a typical rectoscope is approximately 25 cm long.

The prior art anoscope is basically a tubular body which is open in both ends, and furnished with a handle. In use, the tube is inserted into the anus, whereupon the physician may visually inspect the anus and extract tissue samples (in a biopsy procedure) with a suitable tool inserted through the lumen in the tubular body.

The prior art rectoscope is similar to the anoscope (but longer), but may in addition have an end cap on that end of the tubular body which is not inserted into the rectum. The cap may be transparent. A pump, usually a rubber bulb which is squeezed to generate a pressure, is connected to the tubular body via a hose. In use, when the rectoscope is inserted into rectum, the rubber bulb is squeezed to increase the pressure inside the cavity which is formed by the tubular body (closed off at the outer end by said cap) and the volume inside the rectum, whereby the rectum wall is dilated. The dilated rectum wall facilitates visual inspection and biopsy.

One disadvantage associated with the known anoscopes and endoscopes is that instruments are introduced through the tubular body by visual guidance. The visibility is limited, and the instruments limit the visibility even more. Another disadvantage associated with prior art rectoscope, is that the pressure and hence dilation of the rectum (generated by the rubber bulb as described above) is compromised when an instrument is inserted through the tubular body (and the end cap is removed). It is therefore a need for an improved anoscope, rectoscope, vaginoscope and amnioscope.

### SUMMARY OF THE INVENTION

The invention is set forth and characterized in the main claim, while the dependent claims describe other characteristics of the invention.

It is thus provided a rigid endoscope having a rigid tubular body with first and second ends and a through-going bore through which an instrument may be introduced to emerge from an opening at a first end of the bore, characterized by an observation channel having a portion with a closed end arranged at a distance behind said opening, and an open end, said observation channel configured for accommodating at least an optical instrument, and the portion of the observation channel is arranged completely or partially within the tubular body circumference or completely or partially within the bore.

In one embodiment, the closed end is arranged in the vicinity of said opening and second end.

The observations channel makes it possible to have a camera integrated in a disposable endoscope with a closed channel where the camera is protected against contamination.

In one embodiment, the closed end is made of a transparent material. The closed end may comprise an optical lens. The open end is in one embodiment configured for connection to a protective sleeve.

In one embodiment, the endoscope is made of a plastic material and is intended for being discarded after use. In one embodiment, the rigid endoscope further comprises an end cap configured for sealable and releasable connection to the observation channel open end.

An obdurator will also be used.

It is also provided a rigid endoscope system, characterized by the rigid endoscope according to the invention; an optical instrument configured for insertion into said observation channel and arranged in the vicinity of said closed end; said optical instrument being operationally connected to a power pack, a wireless transmitter and a control unit; said power pack, wireless transmitter and control unit being configured for insertion into said observation channel. The optical instrument may be a camera, preferably also having a light source. The power pack (21) may be a rechargeable battery pack, and may additionally comprises an induction charging unit. The wireless transmitter may comprise Wi-Fi unit.

The rigid endoscope may be an anoscope, a rectoscope, a vaginoscope or an amnioscope. The rigid endoscope is suitable for being discarded after use.

### BRIEF DESCRIPTION OF THE DRAWINGS

These and other characteristics of the invention will become clear from the following description of embodiments, given as non-restrictive examples, with reference to the attached schematic drawings, wherein:

FIG. 1 is a sketch of an embodiment of the endoscope according to the invention;

FIG. 2 is a front view of the endoscope illustrated in FIG. 1;

FIG. 3 corresponds to FIG. 1, and shows in addition a camera and light introduced into the observation channel;

FIG. 4 corresponds to FIG. 3, and shows in addition a biopsy tool introduced through the endoscopy bore;

FIG. 5 is a sketch of a system utilizing the endoscope according to the invention;

FIG. 6 is a side view of another embodiment of the endoscope according to the invention, with an obdurator installed in the tubular bore;

FIG. 7 is a sectional side view of the embodiment illustrated in FIG. 6, with the obdurator removed;

FIG. 8 is a perspective sectional view of the embodiment illustrated in FIG. 6, with the obdurator removed; and

FIG. 9 is a perspective view of the embodiment illustrated in FIG. 6, with the obdurator installed in the tubular bore.

### DETAILED DESCRIPTION OF AN EMBODIMENT

The following description will use terms such as “horizontal”, “vertical”, “lateral”, “back and forth”, “up and down”, “upper”, “lower”, “inner”, “outer”, “forward”, “rear”, “behind”, “in front of”, etc. These terms generally refer to the views and orientations as shown in the drawings and that are associated with a normal use of the invention. The terms are used for the reader's convenience only and shall not be limiting.

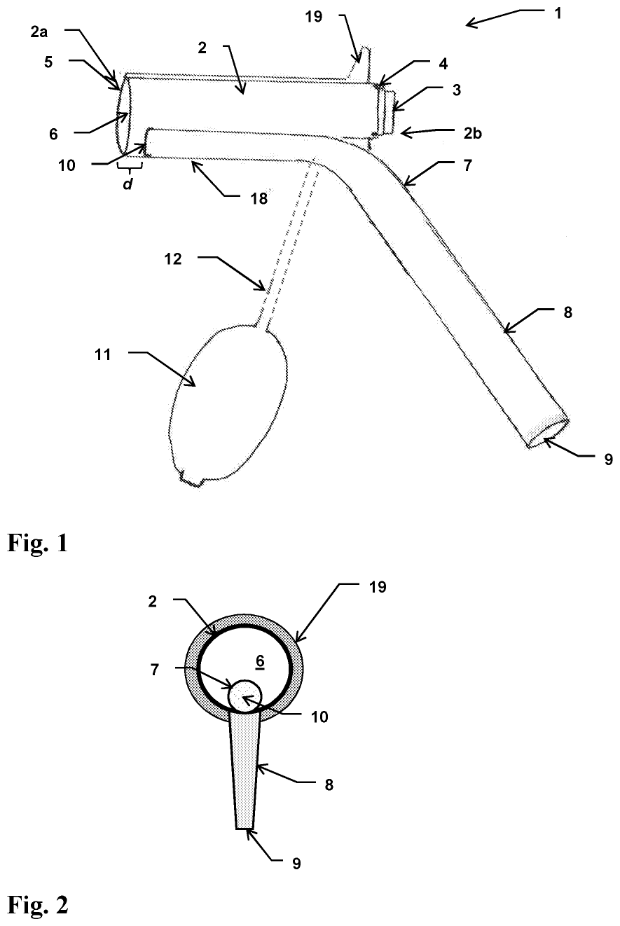

FIG. 1 illustrates an embodiment of the endoscope 1 according to the invention, having a tubular body 2 with a first end 2a and a second end 2b. For the purpose of this description, and in keeping with common terminology in the field of use, the first end 2a will also be referred to as the distal end and the second end 2b will also be referred to as the proximal end. A collar 19 is arranged near the proximal end 2b. For the purpose of this description, the term “endoscope” shall mean a rigid endoscope, and more specifically, an anoscope, a rectoscope, a vaginoscope or an amnioscope. The endoscope 1 shown in FIG. 1 may therefore be an anoscope or a rectoscope, one difference between the two being the length of the tubular body 2, as discussed above. In the case of a rectoscope, an end cap 3 and corresponding seal 4 are arranged at the proximal end 2b to seal off that end of the tubular body which is not inserted into the rectum. Also, a rubber bulb 11 may be connected to the tubular body 2 bore 6 via a hose 12 (drawn in dotted lines to indicate optional feature). In use, the rubber bulb 11 is squeezed to increase the pressure in the cavity which is formed by the tubular body bore 6 and closed off at the proximal end by the end cap 3, whereby the rectum wall is dilated.

The invented endoscope 1 comprises in the illustrated embodiment an observation channel 7 having a portion 18 extending along the tubular body bore 6 and having a closed end 10 in a region in the vicinity of the bore opening 5. The closed end 10 is formed of a transparent material and may comprise a lens (not shown per se). An outer portion of the observation channel 7 is bent with respect to the portion 18 and may conveniently be used as a handle 8 The observation channel 7 comprises in the embodiment illustrated in FIG. 1 an open end 9, into which a camera, light source, etc., may be inserted. In the embodiment shown in FIGS. 1 and 2, the portion 18 of the observation channel is arranged inside the bore 6. Having the portion 18 generally within the bore 6 circumference and generally within the tubular body 2, provides for a slender and compact design. The observation channel material is preferably a light-weight, plastic material, and may be transparent or translucent.

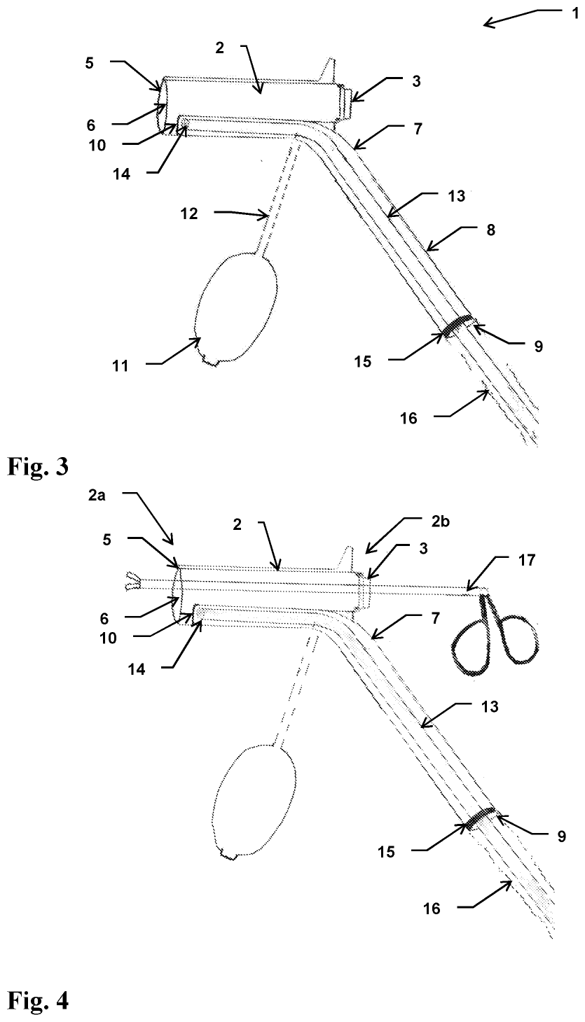

In FIG. 3 an optical instrument 13 has been inserted into the observation channel 7 (a part of which may also be used as a handle 8). Such optical instruments, which may comprise a camera (and light source) 14, are well known in the art and need therefore not be described in more detail here. It will suffice to mention that the optical instrument 13 is inserted a sufficient distance into the observation channel 7 such that the camera 14 is arranged near or at the closed end 10.

The closed end 10 terminates a distance d behind the distal end 2a. The distance d may for example be between 2 and 5 cm, but the invention shall not be limited to such dimensions. One advantageous effect of arranging the closed end 10 a distance d behind (i.e. receded from) the distal end 2a, is that the view of the tissue to be examined (by the optical instrument) is not obstructed. In use, the tissue in the body cavity to be examined is dilated by the distal end 2b, providing an unobstructed view from the closed end 10.

A protective sleeve 16, for example a plastic tube, is in the embodiment illustrated in FIGS. 3 and 4 arranged around that part of the optical instrument 13 which is not inside the observation channel 7, and is sealed around the observation channel 7 open end via a seal ring (e.g. an elastic band) 15.

FIG. 4 illustrates one possible use of the invented endoscope 1, with the optical instrument 13 installed in the observation channel 7, and an instrument (e.g. a biopsy tool) 17 inserted through the endoscopy bore 6. The physician may thus perform a procedure inside the anus, rectum or vagina by operating the instrument 17 through the bore 6, while visually observing the procedure through the optical instrument 13 (and camera 14) installed in the observation channel 7. The closed end 10 and the protective sleeve 16 prevent contamination of the optical instrument 13, and thus render disinfection before use in another procedure unnecessary.

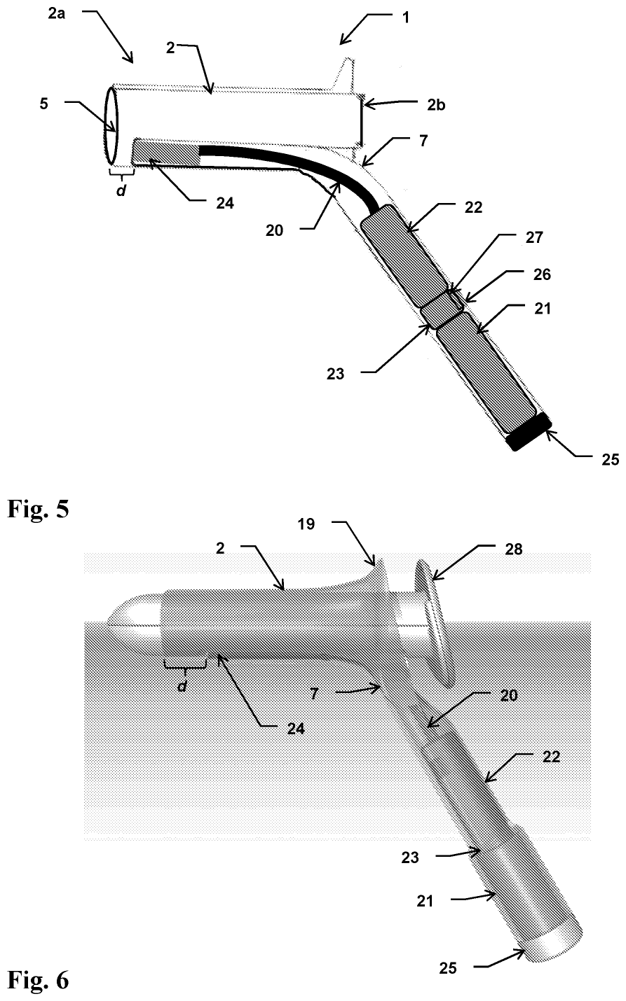

Referring now to FIG. 5, the invented rigid endoscope 1 may be combined with a camera (and preferably an LED light source) 24, configured for insertion into the observation channel 7, near the closed end 10. The camera 24 is connected via a cable 20 to a wireless transmitter 22 (e.g. Wi-Fi, Bluetooth, or similar), a control unit 23 and a power pack 21 (internal connections not illustrated). The power pack 21 may comprise one or more rechargeable batteries (e.g. to be recharged by induction charging). The power pack 21, control unit 23 and wireless transmitter 22 are configured for complete insertion into the observation channel 7. A removable end cap 25 secures and seals this assembly inside the observation channel and effectively seals the observation channel. The control unit 23 may comprise a device by means of which the camera 24 may be operated. Such device may for example be a control knob 27 on the control unit 23, operable by pressing a flexible portion 26 on the observation channel wall. The camera may also be operated by wireless remote control, as the skilled person will understand.

In the embodiment illustrated in FIG. 5, the closed end 10 terminates in the vicinity of, but a distance d behind, the distal end 2a, as explained above with reference to FIG. 1.

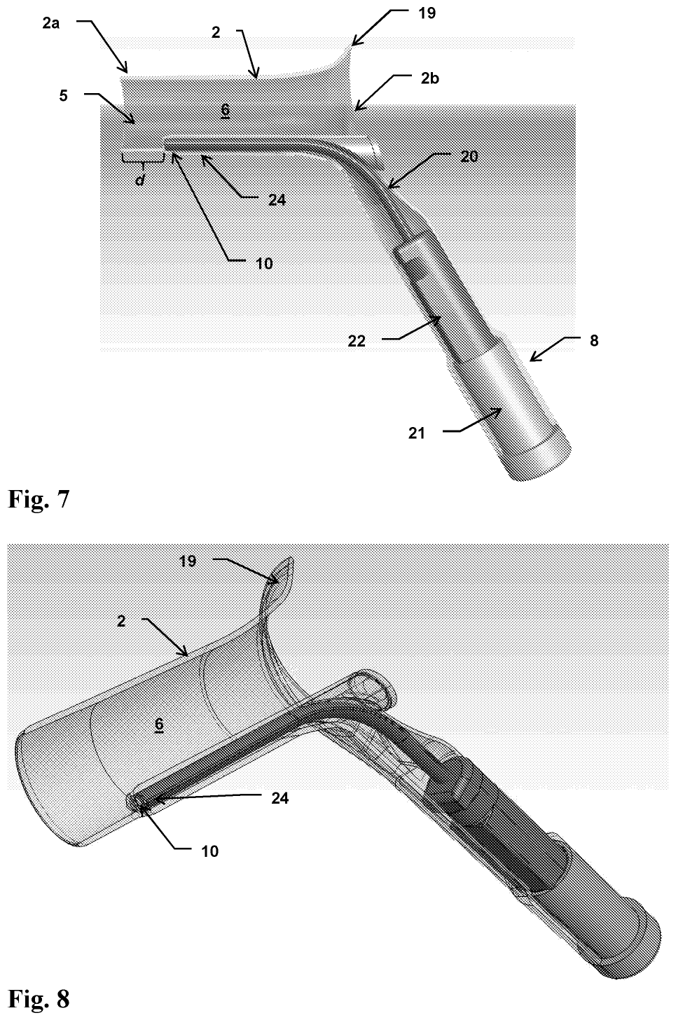

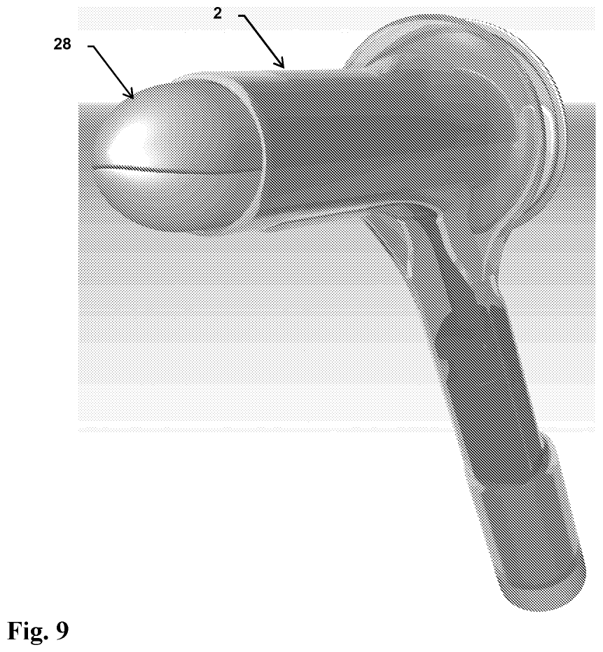

FIGS. 6-9 provide further illustrations of the invented rigid endoscope, of which FIGS. 6 and 9 also illustrate an obdurator 28.

In a practical application, the invented anoscope, rectoscope, vaginoscope or amnioscope may be disposable, while the camera may be used again but without any need for disinfection (other than being swiped with a cloth stained with an alcohol) because it is arranged in a compartment (the observation channel 7) which is sealed off from the tubular body bore 6. In the case of a rectoscope, it is possible to perform procedures with a dilated rectum wall.

1. A rigid endoscope having a rigid tubular body with first and second ends and a through-going bore through which an instrument may be introduced to emerge from an opening at a first end of the bore,
characterized by
an observation channel having:
a portion with a closed end arranged at a distance (d) behind said opening,
an open end, and
an end cap configured for sealable and releasable connection to the open end,
said observation channel configured for accommodating at least an optical instrument operably connected to an assembly comprising a power pack, a wireless transmitter, and a control unit configured for insertion into said observation channel, wherein the end cap secures and seals said assembly inside the observation channel; and
the portion of the observation channel is arranged completely or partially within a circumference of the tubular body, or is arranged completely or partially within the bore.

2. The rigid endoscope of claim 1, wherein the closed end) is arranged in the vicinity of said opening and the second end of the tubular body.

3. The rigid endoscope of claim 1, wherein the closed end is made of a transparent material.

4. The rigid endoscope of claim 1, wherein the closed end comprises an optical lens.

5. The rigid endoscope of claim 1, wherein the open end is configured for connection to a protective sleeve.

6. The rigid endoscope of claim 1, wherein the endoscope is made of a plastic material and is intended for being discarded after use.

7. (canceled)

8. The rigid endoscope (1) of claim 1, wherein the endoscope is an anoscope, a rectoscope, a vaginoscope, or an amnioscope.

9. (canceled)

10. The rigid endoscope of claim 1, wherein the optical instrument comprises a camera.

11. The rigid endoscope of claim 1, wherein the power pack comprises at least one of: (i) a rechargeable battery pack, or (ii) an induction charging unit.
